(19) United States
(12) Patent Application Publication
(10) Pub. No.: US 2019/0362833 A1
(43) Pub. Date: Nov. 28, 2019

(54) METHOD FOR MEDICAL TREATMENT PLANNING AND A SYSTEM THEREOF

(71) Applicants: Erez Halahmi, Givat Yearim (IL); Natalie Kalev-Kronik, Tel-Aviv (IL)

(72) Inventors: Erez Halahmi, Givat Yearim (IL); Natalie Kalev-Kronik, Tel-Aviv (IL)

(21) Appl. No.: 16/462,540

(22) PCT Filed: Nov. 20, 2017
(86) PCT No.: PCT/IL2017/051266

§ 371 Date: May 21, 2019

Related U.S. Application Data
(60) Provisional application No. 62/425,188, filed on Nov. 22, 2016.

(30) Foreign Application Priority Data
Nov. 22, 2016 (US) 62425188

(51) Int. Cl. G16H 20/40; G16H 10/60

(52) U.S. Cl. CPC G16H 20/40; G16H 10/60

(57) ABSTRACT

The present invention relates to a method and a system for planning a medical treatment for a patient comprising determining a treatment profile to be administered to the patient including a plurality of doses of different amounts, wherein the treatment profile has a certain time pattern.

### TECHNOLOGICAL FIELD

The present invention is in the field of medical treatment, in autoimmune diseases such as cancer treatment, as well as in preventing organ transplantation rejection etc.

### BACKGROUND

The immune system is comprised of many components that are designed to fight infections and malignancies. A fine balance of the immune components controls an immune response to create inflammation and destroy an invading pathogen, an infected cell, or a malignancy. The immune system is also scrutinized so that it does not attack the self and such that at the end of an inflammation period, after the invader/pathogen has been destroyed, the immune reaction subsides.

It is common understanding that malignancies and autoimmune diseases are a result of imbalances in the immune system. While in malignancy, the tumor manipulates the immune system into anergy towards it, and in an autoimmune disorder the components of the immune system (mainly T-cells), that should have been anergic towards the host self-antigens, are activated. For example, after organ transplantation, patients are required to take immune suppressants to prevent organ rejection. For example, Japanese patent No. 4,974,892 describes a method which aims at eliminating the immune cells that cause this rejection, by targeting them specifically.

Adoptive cellular immunotherapy uses infusions of antigen targeted T-cells. The therapeutic tumor-infiltrating lymphocytes (TIL) therapy uses lymphocytes isolated from cancer metastasis which are grown in-vitro with the aid of interleukin-2. The cells are expended to a large quantity by ex-vivo laboratory techniques, and infused back to the patient in one bolus treatment. However, there is no consensus on the useful dosage. Usually, the dose given is the entire stock that has been produced for a specific patient. In some cases there have been trials to administer a given dose at a given schedule, yet the dose used is not intentionally modified between administrations in order to achieve an improved or optimized result. An example of this method is described in U.S. Pat. No. 7,015,205. Moreover, it is important to note that in any cellular immunotherapy, the amount of T-cell produced is a limited resource. In the field of oncology the success rate of cellular immunotherapy is below 16%. With respect to organ transplant, transplant rejection occurs when a transplanted organ is rejected by the immune system, which destroys the transplanted organ. Transplant rejection can be lessened by use of immunosuppressant drugs after transplant. However, patients are required to take immune suppressing medication for the rest of their lives to prevent organ rejection.

### GENERAL DESCRIPTION

Natural biological processes have a characteristic time pattern with a functional shape profile and usually increase or decrease in response to a stimulation. According to this invention, a method of using doses in a medical treatment imitating natural biological processes is proposed and thus greatly improves patient response. The inventors have found that determining a medical treatment protocol having a time pattern imitating the time pattern of the immune system enables to significantly increase the efficacy of the treatment. According to an aspect of the present invention, there is provided a method for planning/managing a medical treatment which comprises determining a treatment profile (e.g. protocol) to be administered to the patient including a plurality of doses of different amounts, wherein the treatment profile has a certain time pattern. The time pattern has a shape profile corresponding to a characteristic time pattern of the immune system. The method may thus determine the treatment profile by fitting the treatment profile to have at least one of the following shape profiles: exponential function, power function, polynomial function, a first degree polynomial, and a partial or complete bell-shape in linear or exponential scale with varying doses over time.

According to another aspect of the present invention, there is provided a method and a system for a medical treatment planning comprising determining a treatment profile to be administered to the patient including a plurality of doses, wherein the treatment profile has a time pattern which may be defined to have a period not exceeding two weeks. In this case, the treatment profile may have doses of similar amounts. Moreover, the interval between repeated administrations of the treatment profile may be constant. For example, the periodic interval between two doses may be in the range of about one to six days. As used herein the term “about” refers to plus or minus 10 percent. The treatment profile may be administered in any conventional way such as by injection, infusion (into tissue/blood), orally, dermally, by using ophthalmic, otologic, nasal, urogenital or rectal (enteral) routes. The method may thus determine the treatment profile by fitting the treatment profile to have at least one of the following shape profiles: exponential function, power function, polynomial function, a first degree polynomial, and a partial or complete bell-shape in linear or exponential scale with varying doses over time.

In some embodiments, the method comprises managing a medical treatment involving an individual's immune system comprising at least one of the following: cancer treatment, organ transplantation rejection treatment, and autoimmune treatment.

In some embodiments, the method may thus determine the treatment profile by generating the time pattern by planning doses of different amounts having a certain period in a range of about one to six days. More specifically, the method may determine the treatment profile by planning more than one cycle of treatment with varying doses of lymphocytes or therapeutic cells. In particular, the method may provide a treatment of diseases using the application of infusions of immunological cells by applying varying amounts of therapeutic cells (e.g. T-cells) for improved response. The treatment profile may comprise more than one cycle of treatment with therapeutic cells. As described above, in a natural biological process, the amount of T-cells vs. time is bell-shaped. However, in most cases where external boost of the immunological system is required and provided in the form of adoptive cellular immunotherapy, the intervention point in time should start from the peak of the bell. The reason is that usually the point in time where external medical intervention takes place is after the body's bell-shaped reaction has reached its peak, but failed to be effective. External intervention continues from this point in time and boosts the positive reaction. Within the limitations of a given amount of cells produced per patient, the present invention provides a novel method and system that allocates the given amount into different doses.

In the case of an organ transplant, patients are required to take immune suppressing medication for the rest of their lives, to prevent organ rejection. The patient's immune system does not recognize the transplanted organ as “self”. However, there may be a window of opportunities, right after the transplant, when the organ rejecting lymphocytes may be destroyed by injecting special lymphocytes directed against the rejecting cells, in changing doses, and increasing, through variating dose injections, the number of regulatory T cells and other components of the immune system which are responsible for self-recognition.

In diabetes type1, insulin secreting cells on the pancreas are mistakenly identified as non-self and cytotoxic T lymphocytes target them and destroy them. This process takes years. Within this window of opportunity, lymphocytes in variating doses can be injected into the patient to destroy the dangerous self-destroying cytotoxic T cells.

In some embodiments, the method may determine the treatment profile by fitting the treatment profile to have a shape profile with decreasing doses over time. The treatment starts with a high dosage to achieve maximal effect.

In this way, the present invention provides an ability to plan an optimal infusion schedule within the limitations of a given therapeutic-cells reservoir. Simulations show that such a method improves dramatically the chances of response, while reducing the required time as well as the amount of therapeutic cells required.

In some embodiments, the method comprises calculating at least one of the plurality of doses to be decreased by an order of magnitude as compared to at least one dose on the time pattern.

In some embodiments, the present invention shows that, in contrast to the unquantified “the more the better” approach, the ratio of T cell to tumour cell is a key factor.

In some embodiments, the method comprises determining a certain dose of treatment to be administered to be identical for all patients.

Alternatively, the method comprises receiving an input being indicative of a patient's condition by, for example, obtaining a number of pathological cells derived from at least one of the following: CT scan, MRI, any imaging device and a caliper device. The input may comprise a tumor load or an amount of Immunoglobulin E.

In some embodiments, the method comprises calculating the amount of at least one dose to be proportional to the tumor load. The amount of at least one dose may be selected to be at least five times the tumor load. The amount of at least one dose may also selected to be between five times to hundred times the tumor load.

In some embodiments, the method comprises analyzing at least one of the following: a blood sample, and an analysis of a TGF beta measurement.

According to another broad aspect of the present invention, there is provided a system for medical treatment planning comprising a control unit configured and operable to determine a treatment profile to be administered to a patient comprising a plurality of doses of different amounts, wherein the treatment profile has a certain time pattern.

In some embodiments, the control unit determines a treatment profile having a shape profile with decreasing doses over time. The control unit may also determine a treatment profile having at least one of the plurality of doses decreased by an order of magnitude as compared to at least one dose on the time pattern. The control unit may determine a treatment profile having at least one of the following shape profiles: exponential function, power function, polynomial function, a first degree polynomial, and a partial or complete bell-shape in linear or exponential scale with varying doses over time. The control unit may determine a treatment profile including doses of different amounts having a certain period in a range of about one to six days. The control unit may be configured and operable to receive an input being indicative of a patient's condition. The control unit may be configured and operable to calculate the amount of at least one dose to be proportional to the tumor load.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 illustrates a method of medical treatment planning;

FIG. 2 illustrates a system of medical treatment planning;

FIG. 3 illustrates simulation results of the proposed method showing its benefit;

FIG. 4 illustrates simulation results of the number of cancer cells vs. therapeutic cells both normalized by the initial tumor load; and

FIG. 5A-5B illustrate simulation results of the proposed method having different shape profiles according to some embodiments of the present invention.

### DETAILED DESCRIPTION OF EMBODIMENTS

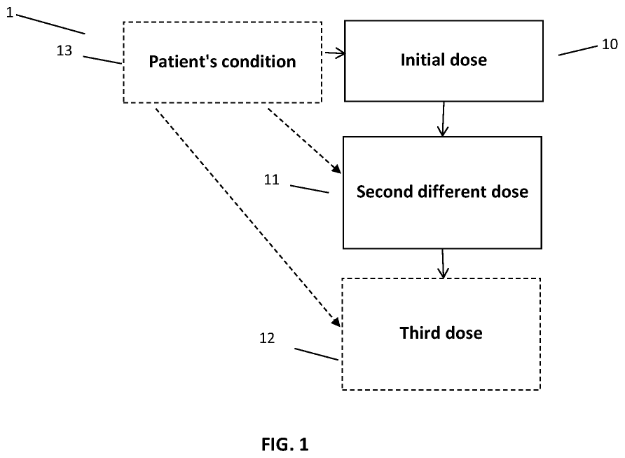

Referring to FIG. 1, there is illustrated, by way of a flow chart diagram, a method 1 of the present invention for performing a medical treatment with at least two cycles of treatment. The first cycle 10 is initially given to the patient. If the treatment is a cancer treatment, the therapeutic cells injected to the patient can be Lymphokine activated killer (LAK), Natural killer (NK) cells, T cells or any other type of therapeutic cells. If the treatment is aimed at preventing organ transplantation rejection, the therapeutic cells injected to the patient can be LAK, NK, cytotoxic T lymphocytes (CTL), or regulatory T cells (Tregs). If the treatment is an autoimmune treatment, the therapeutic cells injected to the patient can be CTL or other type of lymphocyte that can kill rejection CTLs, as well as Tregs to induce anergy.

After the first dose 10 is given, a second dose 11 is given to the patient. According to one embodiment of the present invention, the amount of cells (the dose) in 11 may be different than the dose given in 10. Alternatively, the dose in 11 may be substantially similar to the dose given in 10, but in this case the time interval between the doses does not exceed two weeks, imitating the time pattern of the immune system. The different doses are given according to a certain time pattern, as will be shown further below. For example, the time pattern may be bell shaped over time. In this connection it should be understood that, as described above, natural biological processes, and in particular biological process involving the immunization system, have a characteristic time pattern with a functional shape profile, and usually increase or decrease in response to stimulation. The inventors of the present invention have shown that by using a treatment profile having a characteristic shape corresponding to the kinetics of immunization mechanism, enables to significantly improve the treatment results. Typically, kinetics of various anti-viral mechanisms show that the magnitude of a response or an infection has a certain time pattern. For example, this is illustrated in FIG. 16.8 A, page 362 of Cellular and Molecular immunology 9^{th} ed. 2018 Elsevier. As shown in the figure, the time pattern of these mechanisms comprises the following shape profile: exponential function and/or power function and/or polynomial function and/or a first degree polynomial (e.g. with a first coefficient of 5 or more) and/or and a partial or complete bell-shape in linear or exponential scale.

The treatment profile therefore comprises at least two doses over a predetermined period of time. As long as the patient responds to the treatment, giving a second dose 11 that is different than the first dose 10 can provide much better overall results. An optional third or more doses may be given thereafter. The invention is not limited of the number of doses to be given to the patient. The second dose 11 given after the first dose 10 may be smaller. This reduces the number of required therapeutic cells and may also increase the patient's response as the therapeutic cells do not interrupt each other. The amount of different doses may be determined empirically (according to the amount of therapeutic cells available) or may be determined with respect to a patient's condition as illustrated by optional step 13. The patient's condition 13 may include the magnitude of the pathology and/or of the disorder. The magnitude of the pathology may be evaluated, for example, by measurement or by estimation. This evaluation may serve as input for determining the amount of the initial dose to be used 10. The amount of the doses may thus correspond to the magnitude of the pathology and may resemble a bell-shaped graph in linear or exponential scale. When the treatment starts and the pathology is already significant, then the beginning of the bell-shape is not selected, but rather its peak.

Optionally, the amount of the initial dose (and optionally even of the following doses) is determined with respect to a tumor load of a patient in case the treatment provided is a cancer treatment. The tumor load may be defined as the number of cancer cells. For example, the amount of the first dose is selected to be at least five times the tumor load. The amount of the first dose may be selected to be between five times to a hundred times the tumor load.

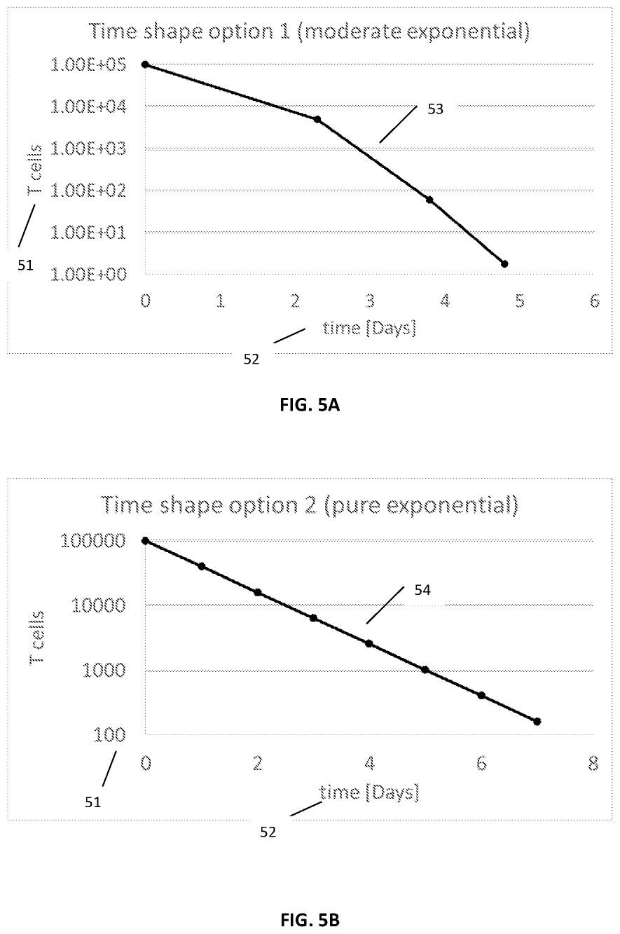

If the treatment provided is an organ transplant rejection prevention, there may be a requirement to eliminate certain cytotoxic lymphocytes and/or inject and support regulatory T cells that prevent rejection and control the cytotoxic lymphocytes by injecting special lymphocytes directed against the rejecting cells, or the number of regulatory T cells or other components of the immune system which are responsible for self-recognition in a similar manner to FIG. 5A illustrated further below.

As will be appreciated by one skilled in the relevant field, the present invention may be, for example, embodied as a computer system, a method, or a computer program product. Accordingly, various embodiments may take the form of an entirely hardware embodiment, an entirely software embodiment, or an embodiment combining software and hardware aspects. Furthermore, particular embodiments may take the form of a computer program product stored on a computer-readable storage medium having computer-readable instructions (e.g., software) embodied in the storage medium. Various embodiments may take the form of web-implemented computer software. Any suitable non-transitory computer-readable storage medium may be utilized including, for example, hard disks, compact disks, DVDs, optical storage devices, and/or magnetic storage devices.

Various embodiments are described below with reference to block diagrams and flowchart illustrations of methods, apparatuses (e.g., systems) and computer program products. It should be understood that each block of the block diagrams and flowchart illustrations, and combinations of blocks in the block diagrams and flowchart illustrations, respectively, can be implemented by a computer executing computer program instructions. These computer program instructions may be loaded onto a general purpose computer, special purpose computer, or other programmable data processing apparatus to produce a machine, such that the instructions which execute on the computer or other programmable data processing apparatus create means for implementing the functions specified in the flowchart block or blocks.

These computer program instructions may also be stored in a computer-readable memory that can direct a computer or other programmable data processing apparatus to function in a particular manner such that the instructions stored in the computer-readable memory produce an article of manufacture that is configured for implementing the function specified in the flowchart block or blocks. The computer program instructions may also be loaded onto a computer or other programmable data processing apparatus to cause a series of operational steps to be performed on the computer or other programmable apparatus to produce a computer implemented process such that the instructions that execute on the computer or other programmable apparatus provide steps for implementing the functions specified in the flowchart block or blocks.

Accordingly, blocks of the block diagrams and flowchart illustrations support combinations of mechanisms for performing the specified functions, combinations of steps for performing the specified functions, and program instructions for performing the specified functions. It should also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and other hardware executing appropriate computer instructions.

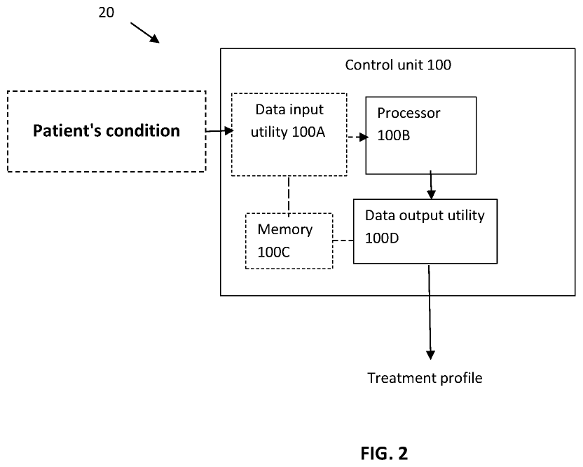

Reference is made to FIG. 2 illustrating, by way of a block diagram, the system 20 of the present invention which may be performed by means of a control unit 100, such as a DSP, microcontroller, FPGA, ASIC, etc., or any other conventional and/or dedicated computing unit/system. The term “control unit” should be expansively construed to cover any kind of electronic device with data processing capabilities, including, by way of non-limiting example, personal computers, servers, computing systems, communication devices, processors (e.g. digital signal processor (DSP), microcontrollers, field programmable gate array (FPGA), application specific integrated circuit (ASIC), etc.) and other electronic computing devices. The control unit 100 may comprise a general-purpose computer processor, which is programmed in software to carry out the functions described hereinbelow. Unless specifically stated otherwise, as apparent from the following discussions, it is appreciated that throughout the specification discussions utilizing terms such as “planning”, “determining”, “analyzing” or the like, refer to the action and/or processes of a computer that manipulate and/or transform data into other data, the data represented as physical, e.g. such as electronic, quantities. Also, operations in accordance with the teachings herein may be performed by a computer specially constructed for the desired purposes or by a general purpose computer specially configured for the desired purpose by a computer program stored in a computer readable storage medium. The software may be downloaded to control unit 100 in electronic form, over a network, for example, or it may alternatively be provided on tangible media, such as optical, magnetic, or electronic memory media. Alternatively or additionally, some or all of the functions of the control unit 100 may be implemented in dedicated hardware, such as a custom or semi-custom integrated circuit or a programmable digital signal processor (DSP). Control unit 100 comprises an optional data input utility 100A including a communication module for receiving a patient's condition, a data output utility 100D for generating data relating to the treatment profile to be administered to the patient, an optional memory (i.e. non-volatile computer readable medium) 100C for storing a database i.e. certain time patterns, and a data processing utility 100B adapted for determining a treatment profile. This process of determining the treatment profile may be run several times during the course of treatment.

A system for medical treatment planning according to various embodiments may comprise one or more central servers and one or more data collection computer devices that are connected to communicate with central servers via any suitable network (e.g., the Internet or a LAN). In particular embodiments, the data collection computer devices may be handheld tablet computers or smartphones that are adapted to communicate with the system's central servers via a wireless network. It should be understood, however, that any other suitable hardware arrangement may be used to implement various embodiments of the systems described below.

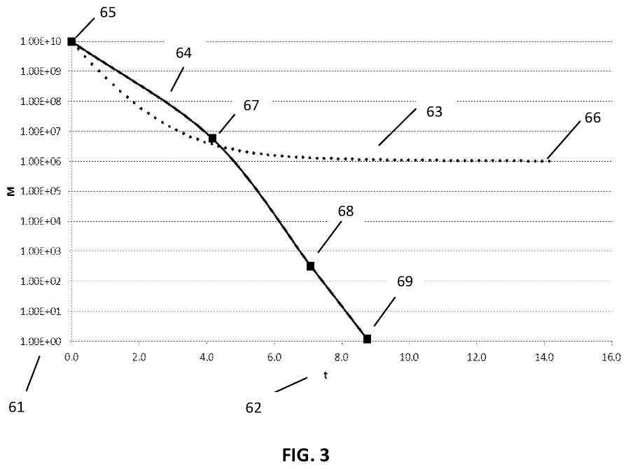

Referring to FIG. 3, a simulation of two cases of treatment of melanoma using T-cells is presented. The vertical axis 61 represents the total number of melanoma cells. The horizontal axis 62 represents the time in days from the point the treatment starts. Graph 63 shows a simulated reaction using a single dose of 1.15E+12 T-cells that was given at the beginning of the treatment 65. Graph 64 shows a simulated reaction using the teachings of the present invention. In the non-optimized case represented by graph 63 after 14 days as shown by point 66, the number of melanoma cells reaches to slightly more than 1.0E+06 cells. If the graph 63 is continued to infinity, the minimum number of melanoma cells that can be reached is slightly below 1.0E+6 cells, which means a recovery is questionable for this patient. However, graph 64 made by using the teachings of the present invention shows a very positive improvement. At the beginning of the treatment 65, a first dose of 1.0E+12 T-cells is given to the patient. This is slightly less than the initial dose in the non-optimized method. After about 4 days as shown by point 67, a second dose of 1.0E+11 T-cells is given to the patient. Therefore, in this non-limiting example, the second dose is smaller than the first dose by an order of magnitude. After about 3 more days (about 7 days from the beginning of the treatment) as shown by point 68, an additional third dose of 5.0E+10 T-cells is given to the patient. So the total number of T-cells given to the patient in the case of graph 64 is identical to the number of T-cells given in the case of graph 63. However, after about 9 days, as shown by point 69 from the beginning of the treatment 65, in the case of graph 64, the patient shows a full recovery. This means that in much less time, a much better result was achieved due to the use of several smaller doses over time. It should be noted that according to numerous simulations performed, the most important point in this method is the repeated varying doses. The exact timing and exact dose is of much less importance. It can also be observed that the shape of the number of T-cells over time in graph 64 corresponds to a bell-shape (under logarithmic scale) starting from its peak, as described above. As clearly shown in the figure, the inventors have demonstrated that a fraction of the same total dose of therapeutic cell into several doses (smaller in this non-limiting example) produced better melanoma decrease. Therefore, the inventors have demonstrated that repeated administrations of therapeutic cells exhibit stronger anti-tumor effect than an equivalent single bolus dose.

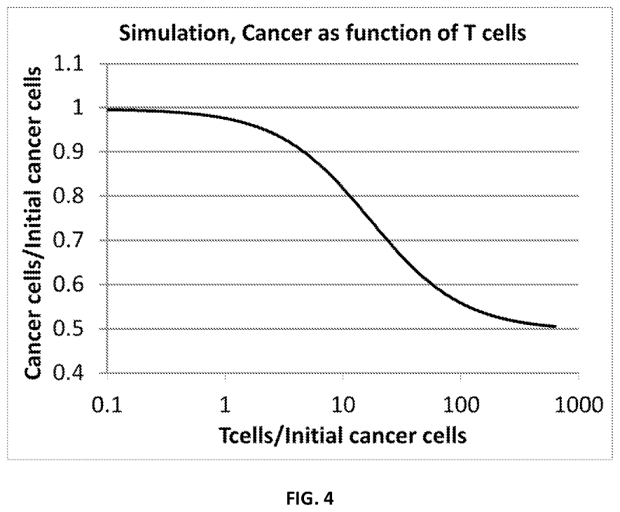

Referring to FIG. 4, this illustrates a simulation of number of cancer cells vs. number of T-cells, both normalized by the initial tumor load, for a single dose. It can be observed that the higher the ratio of therapeutic cells to cancer cells, the sharper the reduction in the relative cancer cell growth or depletion, with the steepest descent at about 10 T cells to 1 cancer cell. The inventors have demonstrated that to obtain an efficient treatment, the range of the amount of the dose may be selected to be between five to a hundred times the numbers of cancer cells.

Referring to FIG. 5A-5B, a simulation of two cases of treatment of melanoma using T-cells is presented. The vertical axis 51 represents the total number of T-cells to be administered. The horizontal axis 52 represents the time in days from the point the treatment starts. Graph 53 shows the simulated reaction using a repeated varying dose of T-cells. Graph 53 has a time pattern having a shape defined by a moderate exponential shape function (or bell shape in logarithmic scale). At the beginning of the treatment, a first dose of 1.00E+05 T-cells is given to the patient with initial 1.00E+4 cancer cells. After about 2.3 days, a second dose of 4.97E+03 T-cells is given to the patient. After about 1.5 more days (about 3.8 days from the beginning of the treatment) an additional third dose of 5.94E+01 T-cells is given to the patient. After about 1 more day (about 4.8 days from the beginning of the treatment) an additional fourth dose of 1.80E+00 T-cells is given to the patient. Although not shown in the figure, the number of cancer cells reaches almost 1, demonstrating efficiency of the treatment method of the present invention. Graph 54 of FIG. 5B shows the simulated reaction using a repeated varying dose of T-cells. Graph 54 has a time pattern having a shape defined by a pure exponential shape function. At the beginning of the treatment, a first dose of 100,000 T-cells is given to the patient with initial 5.00E+4 cancer cells. After about 1 day, a second dose of 40,000 T-cells is given to the patient. After about 1 more day, an additional third dose of 16,000 T-cells is given to the patient. After about 1 more day, an additional fourth dose of 6,400 T-cells is given to the patient. After about 1 more day, an additional fourth dose of 2,560 T-cells is given to the patient. After about 1 more day, an additional fourth dose of 1,024 T-cells is given to the patient. After about 1 more day, an additional fourth dose of 409 T-cells is given to the patient. After about 1 more day, an additional fourth dose of 163 T-cells is given to the patient. Although not shown in the figure, after about 7 days, the number of cancer cells reaches almost 100 demonstrating efficiency of the treatment method of the present invention.

1. A method for medical treatment planning comprising determining a treatment profile to be administered to the patient including a plurality of doses of different amounts, the treatment profile halving a certain time pattern, wherein said determining comprises at least one of fitting the treatment profile to have a shape profile with decreasing doses over time, calculating at least one dose of the plurality of doses such that at least one of the plurality of doses is decreased by an order of magnitude as compared to at least one dose on the time pattern, fitting the treatment profile to have at least one of the following shape profiles: exponential function, power function, polynomial function, a first degree polynomial, and a partial or complete bell-shape in linear or exponential scale with varying doses over time; generating the time pattern by planning doses of different amounts having a certain period in a range of about one to six days; planning more than one cycle of treatment with varying doses of lymphocytes or therapeutic cells; or defining a certain time pattern having a period not exceeding two weeks.

2. (canceled)

3. (canceled)

4. (canceled)

5. (canceled)

6. The method of claim 1, comprising managing a medical treatment involving an individual's immune system comprising at least one of the following: cancer treatment, organ transplantation rejection, and autoimmune treatment.

7. (canceled)

8. The method of claim 1, wherein t therapeutic cells are T-cells, or Lymphokine activated killer (LAK) cells or Natural killer (NK) cells, cytotoxic T lymphocytes (CTL), or regulatory T cells (Tregs).

9. The method of claim 1, further comprising receiving an input being indicative of a patient's condition.

10. The method of claim 9, wherein said receiving an input being indicative of a patient's condition comprises obtaining a number of pathological cells derived from at least one of the following: CT scan, MRI, any imaging device and a caliper device.

11. The method of claim 9, wherein the input comprises a tumor load or an amount of Immunoglobulin E.

12. The method of claim 11, wherein said determining comprises at least one of calculating the amount of at least one dose to be proportional to the tumor load, calculating the amount of at least one dose to be between five times to hundred times the tumor load.

13. (canceled)

14. The method of claim 1, further comprising analyzing at least one of the following: a blood sample, and an analysis of a TGF beta measurement.

15. (canceled)

16. A system for medical treatment planning comprising a control unit configured and operable to determine a treatment profile to be administered to a patient comprising a plurality of doses of different amounts, the treatment profile having a certain time pattern; wherein the treatment profile has at least one of: a shape profile with decreasing doses over time; at least one of the plurality of doses decreased by an order of magnitude as compared to at least one dose on the time pattern; at least one of the following shape profiles: exponential function, power function, polynomial function, a first degree polynomial, and a partial or complete bell-shape in linear or exponential scale with varying doses over time; or the doses of different amounts having a certain period in a range of about one to six days.

17. (canceled)

18. (canceled)

19. (canceled)

20. (canceled)

21. The system of claim 16, wherein said control unit is configured and operable to receive an input being indicative of a patient's condition.

22. The system of claim 21, wherein the input comprises a tumor load.

23. The system of claim 22, wherein said control unit is configured and operable to calculate at least one of the amount of at least one dose to be proportional to the tumor load or the amount of at least one dose to be between five times to a hundred times the tumor load.

24. (canceled)

25. (canceled)

26. A system for medical treatment planning comprising a control unit configured and operable to determine a treatment profile to be administered to a patient comprising a plurality of doses, wherein the treatment profile has a certain time pattern having a period not exceeding two weeks.
